(19) United States
(12) Patent Application Publication
(10) Pub. No.: US 2023/0355584 A1
(43) Pub. Date: Nov. 9, 2023

(54) COMPOSITIONS AND METHODS FOR THE TREATMENT OF LIPID-RELATED DISORDERS

(71) Applicant: Raziel Therapeutics Ltd., Rehovot (IL)

(72) Inventors: Eran BLAUGRUND, Rehovot (IL); Alon BLOOMENFELD, Hod Hasharon (IL); Sharon COHEN VERED, Kfar Sava (IL); Racheli GUETA, Rehovot (IL)

(21) Appl. No.: 18/245,132

(22) PCT Filed: Sep. 15, 2021
(86) PCT No.: PCT/IB2021/000640

§ 371 Date: Mar. 13, 2023

Related U.S. Application Data
(60) Provisional application No. 63/079,195, filed on Sep. 16, 2020.

(51) Int. Cl. A61P 3/04; A61K 31/4045; A61K 31/5575; A61K 9/00; A61K 31/517; A61K 31/203

(52) U.S. Cl. CPC A61K 31/4045; A61K 9/0019; A61K 31/203; A61K 31/517; A61K 31/5575; A61P 3/04

(57) ABSTRACT

The present disclosure provides methods for the treatment of lipid-related disorders. In some embodiments, the method is a method of reducing the quantity of adipose tissue in need thereof, comprising administering a plurality of injections of the pharmaceutical compositions provided herein.

### CROSS REFERENCE

This application claims the benefit of U.S. Provisional Application No. 63/079,195, filed on Sep. 16, 2020, which is incorporated by reference herein in its entirety.

### BACKGROUND OF THE INVENTION

For many overweight individuals, submental fat represents an aesthetic problem. Although localized fat deposits are present in many parts of the body, including the arms, thighs, abdomen, and buttocks, submental fat is particularly noticeable due to its prominent location. Until recently, treatments of submental fat have been limited to invasive surgical procedures such as liposuction, fat excision, and complete neck reconstruction. Surgery is associated with the risks of anesthesia, infection, bleeding, bruising, and scarring, as well as the possibility of poor outcome, discomfort, and prolonged recovery time for the patient. Accordingly, there is a need for nonsurgical alternatives for the treatment of submental fat.

The submental region represents an ideal anatomical area for an injectable treatment for fat reduction. Recently, several treatments have been developed and approved as “non-invasive” treatments for fat reduction. Kybella is an approved treatment for submental fat based on sodium deoxycholate injection. However, due to the onerous treatment regimen (4 to 6 treatments separated by 4-week intervals) and adverse events such as swelling and bruising that repeat after every treatment, there remains a need for the development of new treatments for submental fat.

### SUMMARY OF THE INVENTION

Provided herein, in one aspect, is a method of reducing the quantity of adipose tissue in a subject in need thereof, comprising administering a plurality of injections of a pharmaceutical composition into an adipose portion of the arms, thighs, buttocks, abdomen, or submental region of the subject, wherein the pharmaceutical composition comprises:
a therapeutically effective amount of a compound of Formula (I):

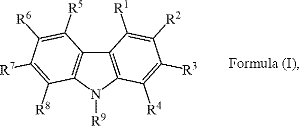

or a pharmaceutically acceptable salt thereof, wherein:
   each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), —ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O);
   R⁹ is C₁₋₉alkyl, C₂₋₉alkenyl, C₂₋₉alkynyl, or a 3- to 10-membered heterocycloalkyl; wherein R⁹ is substituted with at least one quaternary amino group or phosphonium group;
   each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
   each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl which is optionally substituted; and
   each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients.

In some embodiments, said pharmaceutical composition comprises about 10% to about 30% water by weight.

In some embodiments, the plurality of injections are administered to the adipose portion of the buttocks, arms, or thighs of the subject. In some embodiments, the plurality of injections are administered to the adipose portion of the buttocks of the subject. In some embodiments, the plurality of injections are administered to the adipose portion of the arms of the subject. In some embodiments, the plurality of injections are administered to the adipose portion of the thighs of the subject. In some embodiments, the plurality of injections are administered to the adipose portion of the thighs of the abdomen of the subject.

In some embodiments, the plurality of injections is administered to an adipose portion of the submental region of the subject. In some embodiments, the plurality of injections is provided in a specific grid pattern. In some embodiments, each of the plurality of injections into the adipose portion of the subject is provided at a different location of the adipose portion of the subject. In some embodiments, the plurality of injections comprises between 20 and 60 injections. In some embodiments, the plurality of injections comprises between 40 and 50 injections. In some embodiments, the size of the grid is more than 20 points. In some embodiments, the size of the grid is more than 40 points.

In some embodiments, the volume of each injection is between about 25 to about 250 microliters. In some embodiments, the volume of each injection is between about 25 to about 150 microliters. In some embodiments, the volume of each injection is between about 50 to about 100 microliters.

In some embodiments, the concentration of the compound of Formula (I) in the pharmaceutical composition is between about 20 to about 100 milligrams per milliliter. In some embodiments, the concentration of the compound of Formula (I) in the pharmaceutical composition is between about 40 to about 70 milligrams per milliliter. In some embodiments, the concentration of the compound of Formula (I) in the pharmaceutical composition is about 50 milligrams per milliliter.

In some embodiments, the total dose of the compound of Formula (I) per patient per sitting is less than 500 milligrams. In some embodiments, the total dose of the compound of Formula (I) per patient per sitting is less than 400 milligrams. In some embodiments, the total dose of the compound of Formula (I) per patient per sitting is less than 300 milligrams. In some embodiments, the total dose of the compound of Formula (I) per patient per sitting is between about 0.5 milligrams to about 480 milligrams. In some embodiments, the total dose of the compound of Formula (I) per patient per sitting is between about 1 milligram to about 360 milligrams. In some embodiments, the total dose of the compound of Formula (I) per patient per sitting is between about 5 milligrams to about 240 milligrams. In some embodiments, the total dose of the compound of Formula (I) per patient per sitting is between about 10 milligrams to about 180 milligrams. In some embodiments, the total dose of the compound of Formula (I) per patient per sitting is between about 20 milligrams to about 120 milligrams. In some embodiments, the total dose of the compound of Formula (I) per patient per sitting is between about 40 milligrams to about 80 milligrams.

In some embodiments, each injection comprises between about 1 milligram to about 25 milligrams of the compound of Formula (I). In some embodiments, each injection comprises between about 2.5 milligrams to about 15 milligrams of the compound of Formula (I). In some embodiments, each injection comprises between about 5 milligrams to about 10 milligrams of the compound of Formula (I).

In some embodiments, R⁹ is C₁₋₉alkyl substituted with at least one quaternary amino group. In some embodiments, the at least one quaternary amino group is of Formula (V):

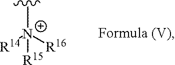

wherein each of R¹⁴, R¹⁵, and R¹⁶ is independently selected from C₁₋₉alkyl, C₂₋₉alkenyl, and C₂₋₉alkynyl. In some embodiments, each of R¹⁴, R¹⁵, and R¹⁶ is independently methyl.

In some embodiments, the at least one ammonium group is a group of Formula (V′):

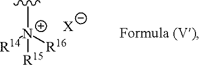

wherein X is a negatively charged ion. In some embodiments, X is a halogen, e.g., Cl.

In some embodiments, each of R¹⁴, R¹⁵, and R¹⁶ is independently C₁₋₉alkyl. In some embodiments, each of R¹⁴, R¹⁵, and R¹⁶ is independently methyl.

In some embodiments, at least one of R¹, R², R³, and R⁴ is halogen. In some embodiments, at least one of R⁵, R⁶, R⁷, and R⁸ is halogen. In some embodiments, at least one of R¹, R², R³, and R⁴ is halogen and at least one of R⁵, R⁶, R⁷, and R⁸ is halogen. In some embodiments, the halogen is bromo.

In some embodiments, at least one of R¹, R², R³, and R⁴ is OH. In some embodiments, at least one of R⁵, R⁶, R⁷, and R⁸ is OH.

In some embodiments, at least one of R¹, R², R³, and R⁴ is nitro and at least one of R⁵, R⁶, R⁷, and R⁸ is nitro.

In some embodiments a composition according to the disclosure comprises at least one active agent selected from the group consisting of:

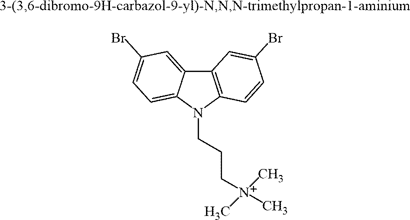

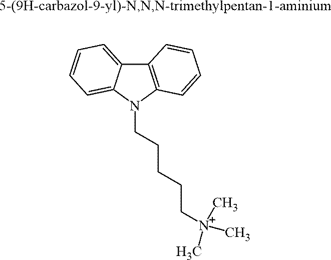

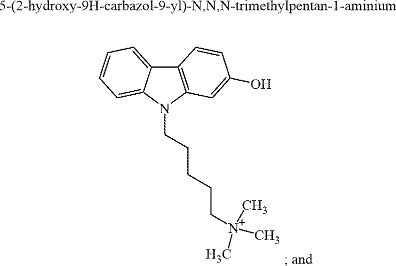

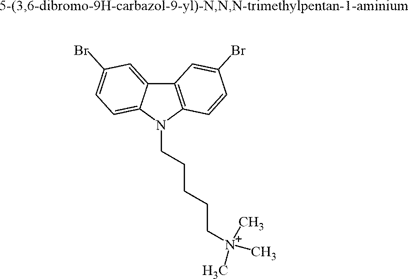

or a pharmaceutically-acceptable salt thereof.

In some embodiments, the compound of Formula (I) is 5-(3,6-dibromo-9H-carbazol-9-yl)-N,N,N-trimethylpentan-1-aminium chloride.

In some embodiments, the pharmaceutical composition comprises about 15% to about 30% water by weight. In some embodiments, the pharmaceutical composition comprises about 15% to about 25% water by weight. In some embodiments, the pharmaceutical composition comprises about 20% to about 30% water by weight. In some embodiments, the pharmaceutical composition comprises about 23% to about 27% water by weight. In some embodiments, the pharmaceutical composition comprises about 24% to about 26% water by weight. In some embodiments, the pharmaceutical composition comprises about 25% water by weight.

In some embodiments, the pharmaceutical composition comprises more than one pharmaceutically acceptable excipient. In some embodiments, the one or more pharmaceutically acceptable excipients is selected from the group consisting of alcohols, fatty acids, ionic surfactants, nonionic surfactants, monoglycerides, diglycerides, triglycerides, esters, antioxidants, amino acids, and amino esters. In some embodiments, the one or more pharmaceutically acceptable excipients is selected from the group consisting of propylene glycol, polyoxyl hydrogenated castor oil, ethanol, glycerol, sorbitol, mannitol, benzyl alcohol, lauryl glucoside, ammonium lauryl sulfate, sodium lauryl sulfate, sodium laureth sulfate, sodium myreth sulfate, docusate, perfluorooctanesulfonate, perfluorobutanesulfonate, sodium stearate, sodium lauroyl sarcosinate, perfluorononanoate, perfluorooctanoate, cetrimonium bromide, cetylpyridinium chloride, benzalkonium chloride, benzethonium chloride, dimethyldioctadecylammonium chloride, dimethyldioctadecylammonium bromide, 3-[(3-cholamidopropyl)dimethylammonio]-1-propanesulfonate, cocamidopropyl hydroxysultaine, cocamidopropyl betaine, phosphatidylserine, phosphatidylethanolamine, phosphatidylcholine, sphingomyelin, octaethylene glycol monododecyl ether, pentaethylene glycol monododecyl ether, nonaethylene glycol, polyethylene glycol nonyl phenyl ether, 2-[4-(2,4,4-trimethylpentan-2-yl)phenoxy]ethanol, Polyoxyl-35 castor oil, polyethoxylated tallow amine, cocamide monoethanolamine, cocamide diethanolamine, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, decyl glucoside, lauryl glucoside, octyl glucoside, lauryldimethylamine oxide, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneisosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, tetratriacontylic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, cervonic acid, herring acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, and citric acid. In some embodiments, the one or more pharmaceutically acceptable excipients is selected from the group consisting of propylene glycol, polyoxyl hydrogenated castor oil, ethanol, glycerol, sorbitol, mannitol, benzyl alcohol, lauryl glucoside, ammonium lauryl sulfate, sodium lauryl sulfate, sodium laureth sulfate, sodium myreth sulfate, sodium stearate, sodium lauroyl sarcosinate, benzalkonium chloride, benzethonium chloride, phosphatidylcholine, sphingomyelin, 2-[4-(2,4,4-trimethylpentan-2-yl)phenoxy]ethanol, Polyoxyl-35 castor oil, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, lauric acid, tridecylic acid, myristic acid, palmitic acid, stearic acid, myristoleic acid, palmitoleic acid, sapienic acid, oleic acid, elaidic acid, vaccenic acid, gadoleic acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, and citric acid. In some embodiments, the one or more pharmaceutically acceptable excipients is selected from the group consisting of ethanol, glycerol, propylene glycol, sorbitol, mannitol, benzyl alcohol, Polysorbate 20, Polysorbate 40, Polysorbate 60, and Polysorbate 80.

In some embodiments, the pharmaceutical composition comprises at least about 0.1% by weight of the compound of Formula (I). In some embodiments, the pharmaceutical composition comprises between about 0.1% to about 10% by weight of the compound of Formula (I). In some embodiments, the pharmaceutical composition comprises between about 1% to about 5% by weight of the compound of Formula (I).

In some embodiments, the pharmaceutical composition further comprises an additional active agent. In some embodiments, the additional active agent is an anti-inflammatory agent, an anti-fibrotic agent, prostaglandin E2, retinoic acid, or halofuginone.

In some embodiments, the pharmaceutical composition is formulated for parenteral injection. In some embodiments, the pharmaceutical composition is formulated for subcutaneous injection. In some embodiments, the pharmaceutical composition is formulated for topical administration.

In some embodiments, the present disclosure provides a pharmaceutical composition comprising:
a therapeutically effective amount of a compound of Formula (I):

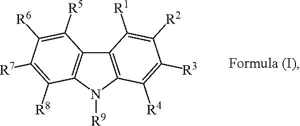

or a pharmaceutically-acceptable salt thereof, wherein:
   each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), —ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O);
   R⁹ is C₁₋₉alkyl, C₂₋₉alkenyl, C₂₋₉alkynyl, or a 3- to 10-membered heterocycloalkyl; wherein R⁹ is substituted with at least one quaternary amino group or phosphonium group;
   each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
   each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl which is optionally substituted; and
   each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients;
optionally wherein said pharmaceutical composition comprises about 10% to about 30% water by weight,
for use in a method of reducing the quantity of adipose tissue in a subject in need thereof, wherein the pharmaceutical composition is in a unit dosage form suitable for administration by a plurality of injections into an adipose portion of the arms, thighs, buttocks, abdomen, or submental region of the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a graphical depiction of the injection and analysis sites used in the distribution study of the various formulations of 5-(3,6-dibromo-9H-carbazol-9-yl)-N,N,N-trimethylpentan-1-aminium chloride.

### DETAILED DESCRIPTION OF THE INVENTION

The present disclosure provides methods of reducing the quantity of adipose tissue in a subject in need thereof, by administering a plurality of injections of a pharmaceutical composition into an adipose portion of the arms, thighs, buttocks, abdomen, or submental region of the subject, wherein the pharmaceutical composition comprises a therapeutically effective amount of a compound of Formula (I), and one or more pharmaceutically-acceptable excipient. In some embodiments, the pharmaceutical composition is injected to an adipose portion of the submental region of the subject. In some embodiments, the pharmaceutical compositions maintain a high exposure of the compound of formula (I) at the injection site versus areas adjacent to the injection site or the plasma, thus minimizing the potential for undesirable side effects at injection-adjacent non-fat tissues, and/or systemic side effects.

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

As used herein the term “about” means an amount within 10% of the stated amount, including the stated amount.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term -C_{x-y}alkylene- refers to a substituted or unsubstituted alkylene chain with from x to y carbons in the alkylene chain. For example -C₁₋₆alkylene- may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, heptyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen, phosphorus, and sulfur atoms may optionally be oxidized and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Aryl” refers to an aromatic ring wherein each of the atoms forming the ring is a carbon atom. Aryl groups can be optionally substituted. Examples of aryl groups include, but are not limited to, phenyl and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-”(such as in “aralkyl”) is meant to include aryl radicals that are optionally substituted.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems wherein at least one of the rings in the ring system is aromatic, i.e., it contains a cyclic, delocalized (4n+2) π-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl is optionally substituted by one or more substituents such as those substituents described herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical may be partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (-) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E- form (or cis- or trans- form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E- forms (or cis- and trans- forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

### Compounds

Provided herein, in one aspect, is a compound of Formula (I):

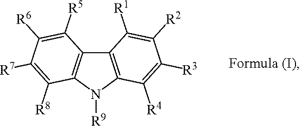

or a pharmaceutically acceptable salt thereof, wherein:
   each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), —ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O);
   R⁹ is C₁₋₉alkyl, C₂₋₉alkenyl, C₂₋₉alkynyl, or 3- to 10-membered heterocycloalkyl; wherein R⁹ is substituted with at least one quaternary amino group or phosphonium group;
   each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
   each R¹¹ and R¹² is independently selected from H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl which is optionally substituted; and
   each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients;
optionally, wherein said pharmaceutical composition comprises about 10% to about 30% water by weight.

In some embodiments, each of R¹, R², R³, R⁴, R⁵, R⁶, R⁷, and R⁸ is independently selected from H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O). In some embodiments, each of R¹, R², R³, R⁴, R⁵, R⁶, R⁷, and R⁸ is independently selected from H, halogen, —CN, —NO₂, -OR¹⁰, -S(=O)₂R¹⁰, -NR¹¹R¹², -C(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, -OR¹⁰, -S(=O)₂R¹⁰, -NR¹¹R¹², -C(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, and -NR¹³S(=O)₂NR¹¹R¹². In some embodiments, each of R¹, R², R³, R⁴, R⁵, R⁶, R⁷, and R⁸ is independently selected from H, halogen, —CN, —NO₂, -OR¹⁰, -NR¹¹R¹², -C(=O)R¹⁰, -C(=O)OR¹⁰, and C₁₋₅alkyl; wherein each alkyl is independently optionally substituted with one or more substituents selected from halogen, —CN, —NO₂, -OR¹⁰, -S(=O)₂R¹⁰, -NR¹¹R¹², -C(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, and -NR¹³S(=O)₂NR¹¹R¹². In some embodiments, each of R¹, R², R³, R⁴, R⁵, R⁶, R⁷, and R⁸ is independently selected from H, halogen, —CN, —NO₂, -OR¹⁰, and -NR¹¹R¹².

In some embodiments, at least one of R¹, R², R³, and R⁴ is halogen. In some embodiments, R¹ is halogen. In some embodiments, R² is halogen. In some embodiments, R³ is halogen. In some embodiments, R⁴ is halogen. In some embodiments, at least one of R⁵, R⁶, R⁷, and R⁸ is halogen. In some embodiments, R⁵ is halogen. In some embodiments, R⁶ is halogen. In some embodiments, R⁷ is halogen. In some embodiments, R⁸ is halogen. In some embodiments, at least one of R¹, R², R³, and R⁴ is halogen and at least one of R⁵, R⁶, R⁷, and R⁸ is halogen. In some embodiments, R¹ is halogen and R⁵ is halogen. In some embodiments, R¹ is halogen and R⁶ is halogen. In some embodiments, R¹ is halogen and R⁷ is halogen. In some embodiments, R¹ is halogen and R⁸ is halogen. In some embodiments, R² is halogen and R⁵ is halogen. In some embodiments, R² is halogen and R⁶ is halogen. In some embodiments, R² is halogen and R⁷ is halogen. In some embodiments, R² is halogen and R⁸ is halogen. In some embodiments, R³ is halogen and R⁵ is halogen. In some embodiments, R³ is halogen and R⁶ is halogen. In some embodiments, R³ is halogen and R⁷ is halogen. In some embodiments, R³ is halogen and R⁸ is halogen. In some embodiments, R⁴ is halogen and R⁵ is halogen. In some embodiments, R⁴ is halogen and R⁶ is halogen. In some embodiments, R⁴ is halogen and R⁷ is halogen. In some embodiments, R⁴ is halogen and R⁸ is halogen. In some embodiments, the halogen is bromo. In some embodiments, the halogen is chloro. In some embodiments, the halogen is fluoro.

In some embodiments, at least one of R¹, R², R³, and R⁴ is OH. In some embodiments, R¹ is OH. In some embodiments, R² is OH. In some embodiments, R³ is OH. In some embodiments, R⁴ is OH. In some embodiments, at least one of R⁵, R⁶, R⁷, and R⁸ is OH. In some embodiments, R⁵ is OH. In some embodiments, R⁶ is OH. In some embodiments, R⁷ is OH. In some embodiments, R⁸ is OH.

In some embodiments, at least one of R¹, R², R³, and R⁴ is nitro and at least one of R⁵, R⁶, R⁷, and R⁸ is nitro. In some embodiments, R¹ is nitro and R⁵ is nitro. In some embodiments, R¹ is nitro and R⁶ is nitro. In some embodiments, R¹ is nitro and R⁷ is nitro. In some embodiments, R¹ is nitro and R⁸ is nitro. In some embodiments, R² is halogen and R⁵ is nitro. In some embodiments, R² is nitro and R⁶ is nitro. In some embodiments, R² is halogen and R⁷ is nitro. In some embodiments, R² is nitro and R⁸ is nitro. In some embodiments, R³ is halogen and R⁵ is nitro. In some embodiments, R³ is nitro and R⁶ is nitro. In some embodiments, R³ is halogen and R⁷ is nitro. In some embodiments, R³ is nitro and R⁸ is nitro. In some embodiments, R⁴ is halogen and R⁵ is nitro. In some embodiments, R⁴ is nitro and R⁶ is nitro. In some embodiments, R⁴ is halogen and R⁷ is nitro. In some embodiments, R⁴ is nitro and R⁸ is nitro.

In some embodiments, R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group. In some embodiments, R⁹ is C₂₋₉alkenyl substituted with at least one phosphonium group. In some embodiments, R⁹ is C₂₋₉alkenyl substituted with at least one quaternary amino group. In some embodiments, R⁹ is C₂₋₉alkynyl substituted with at least one phosphonium group. In some embodiments, R⁹ is C₂₋₉alkynyl substituted with at least one quaternary amino group. In some embodiments, R⁹ is a 3- to 10-membered heterocycloalkyl. In some embodiments, R⁹ is piperazinyl. In some embodiments, R⁹ is pyridinyl. In some embodiments, R⁹ is piperidinyl. In some embodiments, R⁹ is morpholinyl. In some embodiments, R⁹ is thiomorpholinyl. In some embodiments, R⁹ is C₁₋₉alkyl substituted with at least one phosphonium group. In some embodiments, R⁹ is C₁₋₉alkyl substituted with at least one quaternary amino group. In some embodiments, R⁹ is propyl substituted with at least one quaternary amino group. In some embodiments, R⁹ is pentyl substituted with at least one quaternary amino group.

In some embodiments, the at least one quaternary amino group is of Formula (V):

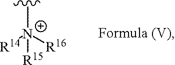

wherein each of R¹⁴, R¹⁵, and R¹⁶ is independently selected from C₁₋₉alkyl, C₂₋₉alkenyl, and C₂₋₉alkynyl. In some embodiments, each of R¹⁴, R¹⁵, and R¹⁶ is independently C₂₋₉alkenyl. In some embodiments, each of R¹⁴, R¹⁵, and R¹⁶ is independently C₂₋₉alkynyl. In some embodiments, each of R¹⁴, R¹⁵, and R¹⁶ is independently C₁₋₉alkyl. In some embodiments, each of R¹⁴, R¹⁵, and R¹⁶ is methyl.

In some embodiments, Formula (V) further comprises a counter-ion, as is represented by the structure of formula (V′):

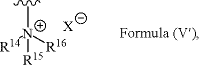

wherein X^{⊝} is a negatively charged counter-ion as defined herein. In some embodiments, X is halogen, e.g., Cl, Br, F, I. In one embodiment, X is a halide, e.g., Cl.

In some embodiments, the at least one phosphonium group is of Formula (VI):

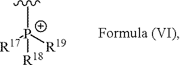

wherein each of R¹⁷, R¹⁸, and R¹⁹ is independently selected from C₁₋₉alkyl, C₂₋₉alkenyl, and C₂₋₉alkynyl. In some embodiments, each of R¹⁷, R¹⁸, and R¹⁹ is independently C₂₋₉alkenyl. In some embodiments, each of R¹⁷, R¹⁸, and R¹⁹ is independently C₂₋₉alkynyl. In some embodiments, each of R¹⁷, R¹⁸, and R¹⁹ is independently C₁₋₉alkyl. In some embodiments, each of R¹⁷, R¹⁸, and R¹⁹ is methyl.

In some embodiments, Formula (VI) further comprises a counter-ion, as is represented by the structure of formula (VI′):

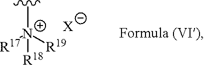

wherein X^{⊝} is a negatively charged counter-ion as defined herein. In some embodiments, X is halogen, e.g., Cl, Br, F, I. In one embodiment, X is a halide, e.g., Cl.

In some embodiments, each R¹⁰ is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, each R¹⁰ is independently selected from H, C₁₋₅alkyl, C₁₋₅heteroalkyl, C₁₋₅haloalkyl, and C₃₋₆cycloalkyl. In some embodiments, each R¹⁰ is independently selected from H, C₁₋₅alkyl, and C₃₋₆cycloalkyl. In some embodiments, each R¹⁰ is H. In some embodiments, each R¹⁰ is independently C₁₋₅alkyl. In some embodiments, each R¹⁰ is independently C₂₋₅alkenyl. In some embodiments, each R¹⁰ is independently C₂₋₅alkynyl. In some embodiments, each R¹⁰ is independently C₁₋₅heteroalkyl. In some embodiments, each R¹⁰ is independently C₁₋₅haloalkyl. In some embodiments, each R¹⁰ is independently C₃₋₆cycloalkyl.

In some embodiments, each R¹¹ and R¹² is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl; or an R¹¹ and an R¹² may be taken together along with the nitrogen atom to which they are attached to form a 3- to 10-membered heterocycloalkyl, which is optionally substituted. In some embodiments, each R¹¹ and R¹² is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, each R¹¹ and R¹² is independently selected from H, C₁₋₅alkyl, C₁₋₅heteroalkyl, C₁₋₅haloalkyl, and C₃₋₆cycloalkyl. In some embodiments, each R¹¹ and R¹² is independently selected from H, C₁₋₅alkyl, and C₃₋₆cycloalkyl. In some embodiments, each R¹¹ and R¹² is H. In some embodiments, each R¹¹ and R¹² is independently C₁₋₅alkyl. In some embodiments, each R¹¹ and R¹² is independently C₂₋₅alkenyl. In some embodiments, each R¹¹ and R¹² is independently C₂₋₅alkynyl. In some embodiments, each R¹¹ and R¹² is independently C₁₋₅heteroalkyl. In some embodiments, each R¹¹ and R¹² is independently C₁₋₅haloalkyl. In some embodiments, each R¹¹ and R¹² is independently C₃₋₆cycloalkyl. In some embodiments, an R¹¹ and an R¹² are taken together along with the nitrogen atom to which they are attached to form a 3- to 10-membered heterocycloalkyl.

In some embodiments, each R¹³ is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, each R¹³ is independently selected from H, C₁₋₅alkyl, C₁₋₅heteroalkyl, C₁₋₅haloalkyl, and C₃₋₆cycloalkyl. In some embodiments, each R¹³ is independently selected from H, C₁₋₅alkyl, and C₃₋₆cycloalkyl. In some embodiments, each R¹³ is H. In some embodiments, each R¹³ is independently C₁₋₅alkyl. In some embodiments, each R¹³ is independently C₂₋₅alkenyl. In some embodiments, each R¹³ is independently C₂₋₅alkynyl. In some embodiments, each R¹³ is independently C₁₋₅heteroalkyl. In some embodiments, each R¹³ is independently C₁₋₅haloalkyl. In some embodiments, each R¹³ is independently C₃₋₆cycloalkyl.

In some embodiments, the compound of Formula (I) is selected from:
3-(3,6-dibromo-9H-carbazol-9-yl)-N,N,N-trimethylpropan-1-aminium,
5-(9H-carbazol-9-yl)-N,N,N-trimethylpentan-1-aminium,
5-(2-hydroxy-9H-carbazol-9-yl)-N,N,N-trimethylpentan-1-aminium, and
5-(3,6-dibromo-9H-carbazol-9-yl)-N,N,N-trimethylpentan-1-aminium.

In some embodiments, the compound of Formula (I) is 3-(3,6-dibromo-9H-carbazol-9-yl)-N,N,N-trimethylpropan-1-aminium. In some embodiments, the compound of Formula (I) is 5-(9H-carbazol-9-yl)-N,N,N-trimethylpentan-1-aminium. In some embodiments, the compound of Formula (I) is 5-(2-hydroxy-9H-carbazol-9-yl)-N,N,N-trimethylpentan-1-aminium. In some embodiments, the compound of Formula (I) is 5-(3,6-dibromo-9H-carbazol-9-yl)-N,N,N-trimethylpentan-1-aminium.

In some embodiments, the compound of Formula (I) is 3-(3,6-dibromo-9H-carbazol-9-yl)-N,N,N-trimethylpropan-1-aminium. In some embodiments, the compound of Formula (I) is 5-(9H-carbazol-9-yl)-N,N,N-trimethylpentan-1-aminium. In some embodiments, the compound of Formula (I) is 5-(2-hydroxy-9H-carbazol-9-yl)-N,N,N-trimethylpentan-1-aminium. In some embodiments, the compound of Formula (I) is 5-(3,6-dibromo-9H-carbazol-9-yl)-N,N,N-trimethylpentan-1-aminium.

In some embodiments, the compound of formula (I) is in a pharmaceutically-acceptable salt form. The term “salt” or “pharmaceutically-acceptable salt” refers to salts derived from a variety of organic and inorganic counter-ions. When a compound of formula (I) contains one or more positive charges, the counter-ion has the corresponding one or more negative charges, generating a neutral molecule. When a compound of formula (I) contains one or more negative charges, the counter-ion has the corresponding one or more positive charges, generating a neutral molecule.

Pharmaceutically-acceptable acid addition salts can be formed with inorganic acids and organic acids and their corresponding counter-ions. Inorganic acids from which salts can be derived include, for example, hydrochloric acid, hydrobromic acid, sulfuric acid, nitric acid, phosphoric acid, and the like. For example, salts may include a counter anion being a halogen counter-anion such as for example chloride and bromide anions. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically-acceptable base addition salts can be formed with inorganic and organic bases and their corresponding counter-ions. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically-acceptable base addition salt is chosen from ammonium, phosphonium, potassium, sodium, calcium, and magnesium salts.

In some embodiments, Compound (I) is in the form of a salt with an inorganic acid. In some embodiments, Compound (I) is in the form of a salt with a hydrochloric acid (i.e., the counter-ion is chloride (Cl⁻)).

In some embodiments, the compound of Formula (I) is 5-(3,6-dibromo-9H-carbazol-9-yl)-N,N,N-trimethylpentan-1-aminium, represented by the structure of formula 1:

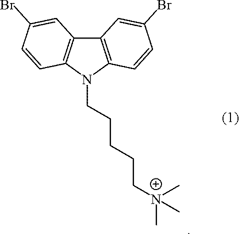

In some embodiments, the compound of Formula (I) is 5-(3,6-dibromo-9H-carbazol-9-yl)-N,N,N-trimethylpentan-1-aminium chloride, represented by the structure of formula 1A:

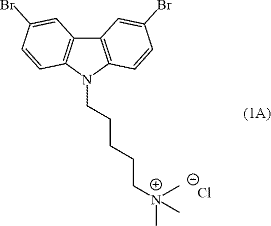 [SMILES: C[N+](C)(C)CCCCCn1c2ccc(Br)cc2c2cc(Br)ccc21 ; [Cl-]]

In some embodiments, the compound of Formula (I) is represented by the structure of formula (2):

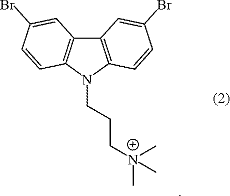

In some embodiments, the compound of Formula (I) is represented by the structure of formula (3):

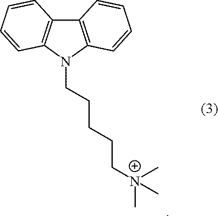

In some embodiments, the compound of Formula (I) is represented by the structure of formula (4):

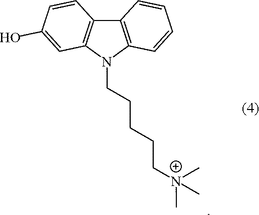

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutical Compositions

A composition of the present disclosure may be formulated in any suitable pharmaceutical formulation. A pharmaceutical composition of the present disclosure typically contains an active ingredient (e.g., a compound of Formula (I), or a pharmaceutically acceptable salt and/or coordination complex thereof), and one or more pharmaceutically acceptable excipients or carriers, including but not limited to: inert solid diluents and fillers, diluents, sterile aqueous solution and various organic solvents, permeation enhancers, solubilizers, and adjuvants. A composition of the present disclosure may be formulated in any suitable pharmaceutical formulation.

Pharmaceutical compositions may be provided in any suitable form, which may depend on the route of administration. In some embodiments, the pharmaceutical composition disclosed herein can be formulated in dosage form for administration to a subject. In some embodiments, the pharmaceutical composition is formulated for parenteral, topical, transdermal, subcutaneous, and/or intraperitoneal administration. In some embodiments, the pharmaceutical composition can be formulated as a unit dosage.

The amount of each compound administered will be dependent on the mammal being treated, the severity of the disorder or condition, the rate of administration, the disposition of the compound and the discretion of the prescribing physician. However, an effective dosage may be in the range of about 0.001 to about 100 mg per kg body weight per day, in single or divided doses. In some instances, dosage levels below the lower limit of the aforesaid range may be more than adequate, while in other cases still larger doses may be employed without causing any harmful side effect, e.g., by dividing such larger doses into several small doses for administration throughout the day. In some embodiments, an effective dosage may be provided in pulsed dosing (i.e., administration of the compound in consecutive days, followed by consecutive days of rest from administration).

In some embodiments, the composition is provided in one or more unit doses. For example, the composition can be administered in 1, 2, 3, 4, 5, 6, 7, 14, 30, 60, or more doses. Such amount can be administered each day, for example in individual doses administered once, twice, or three or more times a day. However, dosages stated herein on a per day basis should not be construed to require administration of the daily dose each and every day. For example, dosage amounts can be administered at a lower frequency, e.g., injection administered every second day to once a month or even longer.

The unit doses can be administered simultaneously or sequentially. The composition can be administered for an extended treatment period. Illustratively, the treatment period can be at least about one month, for example at least about 3 months, at least about 6 months or at least about 1 year. In some cases, administration can continue for substantially the remainder of the life of the subject.

In some embodiments, the pharmaceutical composition comprising the compound of Formula (I) can be administered as part of a therapeutic regimen that comprises administering one or more second agents (e.g. 1, 2, 3, 4, 5, or more second agents), either simultaneously or sequentially with the pharmaceutical composition comprising the compound of Formula (I). When administered sequentially, the pharmaceutical composition comprising the compound of Formula (I) may be administered before or after the one or more second agents. When administered simultaneously, the pharmaceutical composition comprising the compound of Formula (I) and the one or more second agents may be administered by the same route (e.g. injections to the same location), by a different route (e.g. a tablet taken orally while receiving an intravenous infusion), or as part of the same combination (e.g. a solution comprising the pharmaceutical composition comprising the compound of Formula (I) and one or more second agents).

A combination treatment according to the invention may be effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. The exact dosage will depend upon the agent selected, the route of administration, the form in which the compound is administered, the subject to be treated, the body weight of the subject to be treated, and the preference and experience of the attending physician.

In some embodiments, the pharmaceutical composition comprises one or more surfactants. Surfactants which can be used to form pharmaceutical composition and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Polysorbate 40, Polysorbate 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group of vegetable oils, hydrogenated vegetable oils, and triglycerides.

In one embodiment, the composition may include a solubilizer to ensure good solubilization and/or dissolution of the compound of the present disclosure and to minimize precipitation of the compound of the present disclosure. This can be especially important for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. If present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, antifoaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Provided herein, in one aspect, is a pharmaceutical composition comprising:
a therapeutically effective amount of a compound of Formula (I):

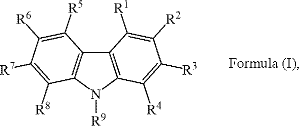

or a pharmaceutically acceptable salt thereof, wherein:
   each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), —ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O)
   R⁹ is C₁₋₉alkyl, C₂₋₉alkenyl, C₂₋₉alkynyl, or a 3- to 10-membered heterocycloalkyl; wherein R⁹ is substituted with at least one quaternary amino group or phosphonium group;
   each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
   each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl which is optionally substituted; and
   each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients;
optionally, wherein said pharmaceutical composition comprises about 10% to about 30% water by weight.

In some embodiments, the pharmaceutical composition comprises about 10% to about 30% water by weight. In some embodiments, the pharmaceutical composition comprises about 15% to about 30% water by weight. In some embodiments, the pharmaceutical composition comprises about 15% to about 25% water by weight. In some embodiments, the pharmaceutical composition comprises about 20% to about 30% water by weight. In some embodiments, the pharmaceutical composition comprises about 23% to about 27% water by weight. In some embodiments, the pharmaceutical composition comprises about 24% to about 26% water by weight. In some embodiments, the pharmaceutical composition comprises about 10% water by weight. In some embodiments, the pharmaceutical composition comprises about 11% water by weight. In some embodiments, the pharmaceutical composition comprises about 12% water by weight. In some embodiments, the pharmaceutical composition comprises about 13% water by weight. In some embodiments, the pharmaceutical composition comprises about 14% water by weight. In some embodiments, the pharmaceutical composition comprises about 15% water by weight. In some embodiments, the pharmaceutical composition comprises about 16% water by weight. In some embodiments, the pharmaceutical composition comprises about 17% water by weight. In some embodiments, the pharmaceutical composition comprises about 18% water by weight. In some embodiments, the pharmaceutical composition comprises about 19% water by weight. In some embodiments, the pharmaceutical composition comprises about 20% water by weight. In some embodiments, the pharmaceutical composition comprises about 21% water by weight. In some embodiments, the pharmaceutical composition comprises about 22% water by weight. In some embodiments, the pharmaceutical composition comprises about 23% water by weight. In some embodiments, the pharmaceutical composition comprises about 24% water by weight. In some embodiments, the pharmaceutical composition comprises about 25% water by weight. In some embodiments, the pharmaceutical composition comprises about 26% water by weight. In some embodiments, the pharmaceutical composition comprises about 27% water by weight. In some embodiments, the pharmaceutical composition comprises about 28% water by weight. In some embodiments, the pharmaceutical composition comprises about 29% water by weight. In some embodiments, the pharmaceutical composition comprises about 30% water by weight.

In some embodiments, the pharmaceutical composition comprises more than one pharmaceutically acceptable excipient. In some embodiments, the one or more pharmaceutically acceptable excipients is selected from alcohols, fatty acids, ionic surfactants, nonionic surfactants, monoglycerides, diglycerides, triglycerides, esters, antioxidants, amino acids, and amino esters. In some embodiments, the one or more pharmaceutically acceptable excipients is selected from ethanol, glycerol, propylene glycol, sorbitol, mannitol, benzyl alcohol, lauryl glucoside, ammonium lauryl sulfate, sodium lauryl sulfate, sodium laureth sulfate, sodium myreth sulfate, docusate, perfluorooctanesulfonate, perfluorobutanesulfonate, sodium stearate, sodium lauroyl sarcosinate, perfluorononanoate, perfluorooctanoate, cetrimonium bromide, cetylpyridinium chloride, benzalkonium chloride, benzethonium chloride, dimethyldioctadecylammonium chloride, dimethyldioctadecylammonium bromide, 3-[(3-cholamidopropyl)dimethylammonio]-1-propanesulfonate, cocamidopropyl hydroxysultaine, cocamidopropyl betaine, phosphatidylserine, phosphatidylethanolamine, phosphatidylcholine, sphingomyelin, octaethylene glycol monododecyl ether, pentaethylene glycol monododecyl ether, nonaethylene glycol, polyethylene glycol nonyl phenyl ether, 2-[4-(2,4,4-trimethylpentan-2-yl)phenoxy]ethanol, Polyoxyl-35 castor oil, polyethoxylated tallow amine, cocamide monoethanolamine, cocamide diethanolamine, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, decyl glucoside, lauryl glucoside, octyl glucoside, lauryldimethylamine oxide, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneisosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, tetratriacontylic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, cervonic acid, herring acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, and citric acid. In some embodiments, the one or more pharmaceutically acceptable excipients is selected from ethanol, glycerol, propylene glycol, sorbitol, mannitol, benzyl alcohol, lauryl glucoside, ammonium lauryl sulfate, sodium lauryl sulfate, sodium laureth sulfate, sodium myreth sulfate, sodium stearate, sodium lauroyl sarcosinate, benzalkonium chloride, benzethonium chloride, phosphatidylcholine, sphingomyelin, 2-[4-(2,4,4-trimethylpentan-2-yl)phenoxy]ethanol, Polyoxyl-35 castor oil, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, lauric acid, tridecylic acid, myristic acid, palmitic acid, stearic acid, myristoleic acid, palmitoleic acid, sapienic acid, oleic acid, elaidic acid, vaccenic acid, gadoleic acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, and citric acid. In some embodiments, the one or more pharmaceutically acceptable excipients is selected from ethanol, glycerol, propylene glycol, sorbitol, mannitol, benzyl alcohol, Polysorbate 20, Polysorbate 40, Polysorbate 60, Polysorbate 80.

In some embodiments, the pharmaceutical composition comprises at least about 0.1% by weight of the compound of Formula (I). In some embodiments, the pharmaceutical composition comprises between about 0.1% to about 10% by weight of the compound of Formula (I). In some embodiments, the pharmaceutical composition comprises between about 1% to about 5% by weight of the compound of Formula (I). In some embodiments, the pharmaceutical composition comprises about 0.1% by weight of the compound of Formula (I). In some embodiments, the pharmaceutical composition comprises about 0.2% by weight of the compound of Formula (I). In some embodiments, the pharmaceutical composition comprises about 0.3% by weight of the compound of Formula (I). In some embodiments, the pharmaceutical composition comprises about 0.4% by weight of the compound of Formula (I). In some embodiments, the pharmaceutical composition comprises about 0.5% by weight of the compound of Formula (I). In some embodiments, the pharmaceutical composition comprises about 0.6% by weight of the compound of Formula (I). In some embodiments, the pharmaceutical composition comprises about 0.7% by weight of the compound of Formula (I). In some embodiments, the pharmaceutical composition comprises about 0.8% by weight of the compound of Formula (I). In some embodiments, the pharmaceutical composition comprises about 0.9% by weight of the compound of Formula (I). In some embodiments, the pharmaceutical composition comprises about 1% by weight of the compound of Formula (I). In some embodiments, the pharmaceutical composition comprises about 1.5% by weight of the compound of Formula (I). In some embodiments, the pharmaceutical composition comprises about 2% by weight of the compound of Formula (I). In some embodiments, the pharmaceutical composition comprises about 2.5% by weight of the compound of Formula (I). In some embodiments, the pharmaceutical composition comprises about 3% by weight of the compound of Formula (I). In some embodiments, the pharmaceutical composition comprises about 3.5% by weight of the compound of Formula (I). In some embodiments, the pharmaceutical composition comprises about 4% by weight of the compound of Formula (I). In some embodiments, the pharmaceutical composition comprises about 4.5% by weight of the compound of Formula (I). In some embodiments, the pharmaceutical composition comprises about 5% by weight of the compound of Formula (I). In some embodiments, the pharmaceutical composition comprises about 5.5% by weight of the compound of Formula (I). In some embodiments, the pharmaceutical composition comprises about 6% by weight of the compound of Formula (I). In some embodiments, the pharmaceutical composition comprises about 6.5% by weight of the compound of Formula (I). In some embodiments, the pharmaceutical composition comprises about 7% by weight of the compound of Formula (I). In some embodiments, the pharmaceutical composition comprises about 7.5% by weight of the compound of Formula (I). In some embodiments, the pharmaceutical composition comprises about 8% by weight of the compound of Formula (I). In some embodiments, the pharmaceutical composition comprises about 8.5% by weight of the compound of Formula (I). In some embodiments, the pharmaceutical composition comprises about 9% by weight of the compound of Formula (I). In some embodiments, the pharmaceutical composition comprises about 9.5% by weight of the compound of Formula (I). In some embodiments, the pharmaceutical composition comprises about 10% by weight of the compound of Formula (I).

In some embodiments, the pharmaceutical composition further comprises an additional active agent. In some embodiments, the additional active agent is selected from an anti-inflammatory agent, an anti-fibrotic agent, prostaglandin E2, retinoic acid, and halofuginone.

In some embodiments, the pharmaceutical composition is formulated for parenteral injection. In some embodiments, the pharmaceutical composition is formulated for subcutaneous injection. In some embodiments, the pharmaceutical composition is formulated for topical administration. In some embodiments, the pharmaceutical composition is formulated for transdermal administration. In some embodiments, the pharmaceutical composition is formulated for intraperitoneal administration.

### Pharmaceutical Compositions for Injection

In some embodiments, the disclosure provides a pharmaceutical composition for injection containing a compound of Formula (I) and a pharmaceutical excipient suitable for injection. Components and amounts of agents in the composition are as described herein.

The forms in which the novel composition of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

The invention also provides kits. The kits may include a pharmaceutical composition comprising a compound of Formula (I) and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Methods of Use

Provided herein, in one aspect, is a method of reducing the quantity of adipose tissue in a subject in need thereof, comprising administering a plurality of injections of a pharmaceutical composition into an adipose portion of the arms, thighs, buttocks, or submental region of the subject, wherein the pharmaceutical composition comprises:
a therapeutically effective amount of a compound of Formula (I):

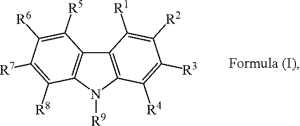

or a pharmaceutically-acceptable salt thereof, wherein:
   each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O);
   R⁹ is C₁₋₉alkyl, C₂₋₉alkenyl, C₂₋₉alkynyl, or a 3- to 10-membered heterocycloalkyl; wherein R⁹ is substituted with at least one quaternary amino group or phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
   each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl; and
   each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients;
optionally, wherein said pharmaceutical composition comprises about 10% to about 30% water by weight.

In some embodiments, the present disclosure provides a pharmaceutical composition comprising:
a therapeutically effective amount of a compound of Formula (I):

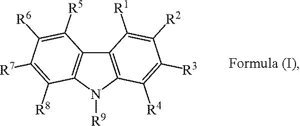

or a pharmaceutically-acceptable salt thereof, wherein:
   each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), —ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O);
   R⁹ is C₁₋₉alkyl, C₂₋₉alkenyl, C₂₋₉alkynyl, or a 3- to 10-membered heterocycloalkyl; wherein R⁹ is substituted with at least one quaternary amino group or phosphonium group;
   each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
   each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl which is optionally substituted; and
   each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients;
optionally wherein said pharmaceutical composition comprises about 10% to about 30% water by weight,
for use in a method of reducing the quantity of adipose tissue in a subject in need thereof, wherein the pharmaceutical composition is in a unit dosage form suitable for administration by a plurality of injections into an adipose portion of the arms, thighs, buttocks, abdomen, or submental region of the subject.

In some embodiments, a plurality of injections are administered according to a predetermined pattern. In some embodiments, the predetermined pattern (such as a grid with points) provides for injections spaced at least 0.5 centimeters, 1 centimeter, 2 centimeters, 3 centimeters, 4 centimeters, 5 centimeters apart. In some embodiments, the plurality of injections is provided in a specific grid pattern. In some embodiments, each of the plurality of injections into the adipose portion of the subject is provided at the same location of the adipose portion of the subject. In some embodiments, each of the plurality of injections into the adipose portion of the subject is provided at a different location of the adipose portion of the subject.

In some embodiments, the plurality of injections comprises between 20 and 60 injections. In some embodiments, the plurality of injections comprises between 40 and 50 injections. In some embodiments, the plurality of injections comprises about 20 injections. In some embodiments, the plurality of injections comprises about 25 injections. In some embodiments, the plurality of injections comprises about 30 injections. In some embodiments, the plurality of injections comprises about 35 injections. In some embodiments, the plurality of injections comprises about 40 injections. In some embodiments, the plurality of injections comprises about 45 injections. In some embodiments, the plurality of injections comprises about 50 injections. In some embodiments, the plurality of injections comprises about 55 injections. In some embodiments, the plurality of injections comprises about 60 injections.

Administrations can be made in a grid pattern with the grid comprising points for injection or regularly spaced doses. Such grids can be in the shape of a square or rectangle. In some embodiments, the size of the grid is more than 20 points. In some embodiments, the size of the grid is more than 40 points. In some embodiments, the size of the grid is about 15 points. In some embodiments, the size of the grid is about 20 points. In some embodiments, the size of the grid is about 25 points. In some embodiments, the size of the grid is about 30 points. In some embodiments, the size of the grid is about 35 points. In some embodiments, the size of the grid is about 40 points. In some embodiments, the size of the grid is about 45 points. In some embodiments, the size of the grid is about 50 points. In some embodiments, the size of the grid is about 55 points. In some embodiments, the size of the grid is about 60 points.

In some embodiments, the volume of each injection is between about 25 to about 250 microliters. In some embodiments, the volume of each injection is between about 25 to about 150 microliters. In some embodiments, the volume of each injection is between about 50 to about 100 microliters. In some embodiments, the volume of each injection is about 25 microliters. In some embodiments, the volume of each injection is about 50 microliters. In some embodiments, the volume of each injection is about 75 microliters. In some embodiments, the volume of each injection is about 100 microliters. In some embodiments, the volume of each injection is about 125 microliters. In some embodiments, the volume of each injection is about 150 microliters. In some embodiments, the volume of each injection is about 175 microliters. In some embodiments, the volume of each injection is about 200 microliters. In some embodiments, the volume of each injection is about 225 microliters. In some embodiments, the volume of each injection is about 250 microliters.

In some embodiments, the concentration of the compound of Formula (I) in the pharmaceutical composition is between about 20 to about 100 milligrams per milliliter. In some embodiments, the concentration of the compound of Formula (I) in the pharmaceutical composition is between about 40 to about 70 milligrams per milliliter. In some embodiments, the concentration of the compound of Formula (I) in the pharmaceutical composition is about 10 milligrams per milliliter. In some embodiments, the concentration of the compound of Formula (I) in the pharmaceutical composition is about 20 milligrams per milliliter. In some embodiments, the concentration of the compound of Formula (I) in the pharmaceutical composition is about 30 milligrams per milliliter. In some embodiments, the concentration of the compound of Formula (I) in the pharmaceutical composition is about 40 milligrams per milliliter. In some embodiments, the concentration of the compound of Formula (I) in the pharmaceutical composition is about 50 milligrams per milliliter. In some embodiments, the concentration of the compound of Formula (I) in the pharmaceutical composition is about 60 milligrams per milliliter. In some embodiments, the concentration of the compound of Formula (I) in the pharmaceutical composition is about 70 milligrams per milliliter. In some embodiments, the concentration of the compound of Formula (I) in the pharmaceutical composition is about 80 milligrams per milliliter. In some embodiments, the concentration of the compound of Formula (I) in the pharmaceutical composition is about 90 milligrams per milliliter. In some embodiments, the concentration of the compound of Formula (I) in the pharmaceutical composition is about 100 milligrams per milliliter.

In some embodiments, the total dose of the compound of Formula (I) per patient per sitting is less than 500 milligrams. In some embodiments, the total dose of the compound of Formula (I) per patient per sitting is less than 400 milligrams. In some embodiments, the total dose of the compound of Formula (I) per patient per sitting is less than 300 milligrams. In some embodiments, the total dose of the compound of Formula (I) per patient per sitting is between about 0.5 milligrams to about 480 milligrams. In some embodiments, the total dose of the compound of Formula (I) per patient per sitting is between about 1 milligram to about 360 milligrams. In some embodiments, the total dose of the compound of Formula (I) per patient per sitting is between about 5 milligrams to about 240 milligrams. In some embodiments, the total dose of the compound of Formula (I) per patient per sitting is between about 10 milligrams to about 180 milligrams. In some embodiments, the total dose of the compound of Formula (I) per patient per sitting is between about 20 milligrams to about 120 milligrams. In some embodiments, the total dose of the compound of Formula (I) per patient per sitting is between about 40 milligrams to about 80 milligrams. In some embodiments, the total dose of the compound of Formula (I) per patient per sitting is about 5 milligrams. In some embodiments, the total dose of the compound of Formula (I) per patient per sitting is about 10 milligrams. In some embodiments, the total dose of the compound of Formula (I) per patient per sitting is about 20 milligrams. In some embodiments, the total dose of the compound of Formula (I) per patient per sitting is about 30 milligrams. In some embodiments, the total dose of the compound of Formula (I) per patient per sitting is about 40 milligrams. In some embodiments, the total dose of the compound of Formula (I) per patient per sitting is about 50 milligrams. In some embodiments, the total dose of the compound of Formula (I) per patient per sitting is about 60 milligrams. In some embodiments, the total dose of the compound of Formula (I) per patient per sitting is about 70 milligrams. In some embodiments, the total dose of the compound of Formula (I) per patient per sitting is about 80 milligrams. In some embodiments, the total dose of the compound of Formula (I) per patient per sitting is about 90 milligrams. In some embodiments, the total dose of the compound of Formula (I) per patient per sitting is about 100 milligrams. In some embodiments, the total dose of the compound of Formula (I) per patient per sitting is about 110 milligrams. In some embodiments, the total dose of the compound of Formula (I) per patient per sitting is about 120 milligrams. In some embodiments, the total dose of the compound of Formula (I) per patient per sitting is about 130 milligrams. In some embodiments, the total dose of the compound of Formula (I) per patient per sitting is about 140 milligrams. In some embodiments, the total dose of the compound of Formula (I) per patient per sitting is about 150 milligrams. In some embodiments, the total dose of the compound of Formula (I) per patient per sitting is about 160 milligrams. In some embodiments, the total dose of the compound of Formula (I) per patient per sitting is about 170 milligrams. In some embodiments, the total dose of the compound of Formula (I) per patient per sitting is about 180 milligrams. In some embodiments, the total dose of the compound of Formula (I) per patient per sitting is about 190 milligrams. In some embodiments, the total dose of the compound of Formula (I) per patient per sitting is about 200 milligrams. In some embodiments, the total dose of the compound of Formula (I) per patient per sitting is about 210 milligrams. In some embodiments, the total dose of the compound of Formula (I) per patient per sitting is about 220 milligrams. In some embodiments, the total dose of the compound of Formula (I) per patient per sitting is about 230 milligrams. In some embodiments, the total dose of the compound of Formula (I) per patient per sitting is about 240 milligrams. In some embodiments, the total dose of the compound of Formula (I) per patient per sitting is about 250 milligrams. In some embodiments, the total dose of the compound of Formula (I) per patient per sitting is about 260 milligrams. In some embodiments, the total dose of the compound of Formula (I) per patient per sitting is about 270 milligrams. In some embodiments, the total dose of the compound of Formula (I) per patient per sitting is about 280 milligrams. In some embodiments, the total dose of the compound of Formula (I) per patient per sitting is about 290 milligrams. In some embodiments, the total dose of the compound of Formula (I) per patient per sitting is about 300 milligrams. In some embodiments, the total dose of the compound of Formula (I) per patient per sitting is about 310 milligrams. In some embodiments, the total dose of the compound of Formula (I) per patient per sitting is about 320 milligrams. In some embodiments, the total dose of the compound of Formula (I) per patient per sitting is about 330 milligrams. In some embodiments, the total dose of the compound of Formula (I) per patient per sitting is about 340 milligrams. In some embodiments, the total dose of the compound of Formula (I) per patient per sitting is about 350 milligrams. In some embodiments, the total dose of the compound of Formula (I) per patient per sitting is about 360 milligrams. In some embodiments, the total dose of the compound of Formula (I) per patient per sitting is about 370 milligrams. In some embodiments, the total dose of the compound of Formula (I) per patient per sitting is about 380 milligrams. In some embodiments, the total dose of the compound of Formula (I) per patient per sitting is about 390 milligrams. In some embodiments, the total dose of the compound of Formula (I) per patient per sitting is about 400 milligrams. In some embodiments, the total dose of the compound of Formula (I) per patient per sitting is about 410 milligrams. In some embodiments, the total dose of the compound of Formula (I) per patient per sitting is about 420 milligrams. In some embodiments, the total dose of the compound of Formula (I) per patient per sitting is about 430 milligrams. In some embodiments, the total dose of the compound of Formula (I) per patient per sitting is about 440 milligrams. In some embodiments, the total dose of the compound of Formula (I) per patient per sitting is about 450 milligrams. In some embodiments, the total dose of the compound of Formula (I) per patient per sitting is about 460 milligrams. In some embodiments, the total dose of the compound of Formula (I) per patient per sitting is about 470 milligrams. In some embodiments, the total dose of the compound of Formula (I) per patient per sitting is about 480 milligrams.

In some embodiments, each injection comprises between about 1 milligram to about 25 milligrams of the compound of Formula (I). In some embodiments, each injection comprises between about 2.5 milligrams to about 15 milligrams of the compound of Formula (I). In some embodiments, each injection comprises between about 5 milligrams to about 10 milligrams of the compound of Formula (I). In some embodiments, each injection comprises about 1 milligram of the compound of Formula (I). In some embodiments, each injection comprises about 2.5 milligrams of the compound of Formula (I). In some embodiments, each injection comprises about 5 milligrams of the compound of Formula (I). In some embodiments, each injection comprises about 10 milligrams of the compound of Formula (I). In some embodiments, each injection comprises about 15 milligrams of the compound of Formula (I). In some embodiments, each injection comprises about 20 milligrams of the compound of Formula (I). In some embodiments, each injection comprises about 25 milligrams of the compound of Formula (I).

### EXAMPLES

### Example 1: Distribution Study of 5-(3,6-Dibromo-9H-Carbazol-9-yl)-N,N,N-Trimethylpentan-1-Aminium Chloride in Various Formulations

Three different formulations of 5-(3,6-dibromo-9H-carbazol-9-yl)-N,N,N-trimethylpentan-1-aminium chloride (Compound 1A) were prepared according to Table 1 below:

TABLE 1

Formulations

| Formulation | Concentration of 5-(3,6-dibromo-9H-carbazol-9-yl)-N,N,N-trimethylpentan-1-aminium chloride | Water Content | Excipients |
|---|---|---|---|
| 1 | 5% | 25% | Propylene glycol 57% Polysorbate 80 10% Benzyl alcohol 3% |
| 2 | 5% | 0% | Cremophor 50% Ethanol 50% |
| 3 | 5% | 60% | Propylene glycol 22% Polysorbate 80 10% Benzyl alcohol 3% |

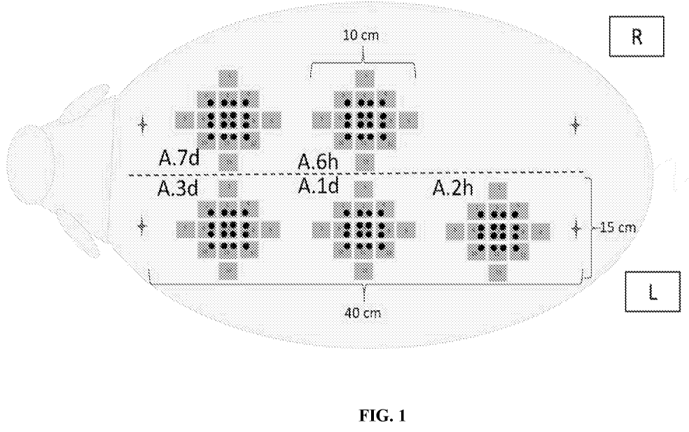

Each formulation was administered to a separate pig via a series of sixteen intra-fat injections comprising 5 mg each of 5-(3,6-dibromo-9H-carbazol-9-yl)-N,N,N-trimethylpentan-1-aminium chloride for a total dose of 80 mg per injection site. Fat cubes were excised, frozen, and analyzed to determine the concentration of 5-(3,6-dibromo-9H-carbazol-9-yl)-N,N,N-trimethylpentan-1-aminium chloride at the injection site, in tissues adjacent to the injection site, and in the plasma at intervals of 2 hours, 6 hours, 1 day, 3 days, and 7 days post-injection. The injection and analysis sites are depicted in FIG. 1. The area under the curve (AUC) data obtained is summarized in Tables 2, 3, and 4 below, and the AUC ratios for the injection site versus adjacent areas and for the injection site versus plasma are summarized in Table 5 below:

TABLE 2

Concentration at Injection Site
Concentration (µg/g)

| Formulation | 2 Hours | 6 Hours | 1 Day | 3 Days | 7 Days | AUC (µg*hr/g) |
|---|---|---|---|---|---|---|
| 1 | 428 | 170 | 135 | 83.6 | 50.0 | 16,028 |
| 2 | 356 | 280 | 164 | 37.2 | 26.1 | 13,491 |
| 3 | 566 | 322 | 334 | 126 | 59.3 | 28,180 |

TABLE 3

Concentration at Areas Adjacent to Injection Site
Concentration (µg/g)

| Formulation | 2 Hours | 6 Hours | 1 Day | 3 Days | 7 Days | AUC (µg*hr/g) |
|---|---|---|---|---|---|---|
| 1 | 57.3 | 27.2 | 44.0 | 28.6 | 17.8 | 4,837 |
| 2 | 33.7 | 11.3 | 57.3 | 10.8 | 11.9 | 3,464 |
| 3 | 172 | 190 | 84.1 | 78.5 | 25.9 | 12,255 |

TABLE 4

Plasma Concentration
Concentration (ng/mL)

| Formulation | 2 Hours | 6 Hours | 1 Day | 3 Days | 7 Days | AUC (ng*hr/mL, [µg*hr/mL]) |
|---|---|---|---|---|---|---|
| 1 | 24.5 | 9.06 | 2.34 | 0.92 | BLQ* | 317 [0.32] |
| 2 | 31.8 | 16.1 | 2.70 | 1.22 | BLQ* | 450 [0.45] |
| 3 | 32.4 | 32.4 | 7.31 | 2.60 | BLQ* | 882 [0.88] |

*BLQ = Below Limit of Quantification

TABLE 5

Area Under the Curve (AUC) Ratios

| Formulation | AUC Ratio (Injection Site /Adjacent Areas) | AUC Ratio (Injection Site /Plasma) |
|---|---|---|
| 1 | 3.31 | 50,600 |
| 2 | 3.89 | 30,000 |
| 3 | 2.30 | 32,000 |

Formulations 1 and 2 display greater ratios of 5-(3,6-dibromo-9H-carbazol-9-yl)-N,N,N-trimethylpentan-1-aminium chloride exposure at the injection site versus areas adjacent to the injection as compared to Formulation 3, thus minimizing the potential for undesirable side effects at injection-adjacent non-fat tissues. Additionally, Formulation 1 displays a greater ratio of 5-(3,6-dibromo-9H-carbazol-9-yl)-N,N,N-trimethylpentan-1-aminium chloride exposure at the injection site versus plasma as compared to Formulations 2 and 3, thus minimizing the potential for undesirable systemic side effects.

### Example 2: Submental Fat Reduction After Dosing of 5-(3,6-Dibromo-9H-Carbazol-9-yl)-N,N,N-Trimethylpentan-1-Aminium Chloride to Human Subjects

28 patients with submental skin fold thickness greater than 1.5 cm as measured with calipers were divided into two cohorts, Cohort 1 (12 patients) and Cohort 2 (16 subjects). In Cohort 1, 8 subjects were injected with a 50 mg per mL solution of 5-(3,6-dibromo-9H-carbazol-9-yl)-N,N,N-trimethylpentan-1-aminium chloride (Compound 1A) in a vehicle comprising water, propylene glycol, and a surfactant, and 4 patients were injected with a placebo solution comprising vehicle only. In Cohort 2, 6 patients were dosed with a placebo solution comprising vehicle only, and 10 patients were injected with a 50 mg per mL solution of 5-(3,6-dibromo-9H-carbazol-9-yl)-N,N,N-trimethylpentan-1-aminium chloride in a vehicle comprising water, propylene glycol, and a surfactant. Dosing is determined by cohort, as shown in Table 6 below:

TABLE 6

Submental Fat Dosing Regimen
N=28

| | Cohort 1 (N=12) | Cohort 2 (N=16) |
|---|---|---|
| Number of subjects - compound/placebo | 8/4 | 10/6 |
| Maximum dose of compound (mg) per vehicle (mL) | Up to 120 mg/2.4 mL/subject | Up to 240 mg/4.8 mL/subject |
| Dose of compound (mg) per vehicle (mL) per single injection | 2.5 mg/0.05 mL/injection | 5 mg/0.1 mL/injection |
| Maximum number of injections | 48 | 48 |

The maximum compound dose was 120 mg per patient (a total of 48 injections at 2.5 mg per injection) for Cohort 1 and 240 mg per patient (a total of 48 injections at 5 mg per injection) for Cohort 2. Injections were given at a 90° angle to the injected skin surface and spaced of a submental area-shaped grid in which the distance between rows was 1 cm and the distance between columns was also 1 cm. Cohort 2 subjects began treatment after Cohort 1 was evaluated for safety on day 28 post-injection.

### Efficacy Results

The MRI data presented in this Phase 2a study demonstrated that the efficacy of Compound 1A in reducing submental fat. This efficacy was evident by changes in both submental thickness and volume. Efficacy was further supported by the clear dose response relationship, with a volume reduction of -22.2% ± 14.9 (P<0.05 vs the placebo group), -10.6% ± 14.5 (P=0.55 vs the placebo group, NS), and -0.4% ± 13.9 for Compound 1A high dose, low dose, and placebo, respectively.

The objective MRI assessments were supported by the Physician Impression scale, which is similar to a Physician Global Assessment (PGS) questionnaire. Physician evaluation of improvement was conducted by using physician’s global assessment of submental fat at post-treatment follow-up visits on Days 28, 56, and 84. The evaluation was made by a physician, based on a comparison of the submental fat area of the subject at each visit time points to the 2D images that were taken at baseline visit, prior to injection of study drug. The assessment graded the improvement in submental fat condition at each follow up on Days 28, 56 and 84 vs. baseline. The improvement scale used in the evaluations was (i) 0: completely clear (no evidence of submental fat volume; 100% improvement); (ii) 1: almost clear (very significant clearance (>90% to <100%); only trace remain); (iii) 2: marked improvement (significant improvement (≥75% to <90%); some evidence of submental fat remains); (iv) 3: moderate improvement (intermediate between slight and marked improvement (≥50% to <75%)); (v) 4: slight improvement (some improvement (≥25% to <50%); significant evidence of submental fat remains); (vi) 5: no change (submental fat has not changed from baseline condition (±25%)); and (vii) 6: worse (submental fat is worse than at baseline evaluation by ≥25% or more).

The Physician Impression scale data showed a more pronounced improvement on Days 56 and 84 compared to Day 28, suggesting that the effects of Compound 1A on submental fat may progress and cumulate in the weeks following the administration of the compound. On Day 84, none of the placebo subjects demonstrated any change in their SMF. In contrast, at the same timepoint, all but one of the 18 Compound 1A -treated subjects demonstrated improvement in submental fat at 84 days after treatment.

Subjects were also evaluated by a Face-Q Questionnaire, at baseline visit prior to injection and at posttreatment follow up visits on Days 28, 56, and 84. The questionnaire was completed by the subjects following a comparison of 2D images taken at each time point and comparison to the images taken at baseline visit, prior to injection of study drug. The comparison of the images at each study visit vs. baseline was done by displaying the images of visit day vs. baseline images on the computer. The FACE-Q questionnaire included a list of 10 questions related to the chin visibility and degree of subject’s satisfaction from his/her chin at each question. Higher sum of the scores reflect a better outcome. A Conversion Table to convert the raw scale summed score into a score from 0 (worst) to 100 (best).

The results of the Subject Face-Q Questionnaire also demonstrated subject satisfaction with Compound 1A. Subjects who were treated with both low- and high-dose Compound 1A showed 2.4-fold and 2.5- fold increase in mean subject satisfaction score, respectively. Subjects treated with placebo did not demonstrate an increased satisfaction score on Day 84 vs. baseline.

Submental fat volume was determined via magnetic resonance imaging (MRI) prior to injection and on day 84 post-injection. Treatment efficacy was evaluated via the Physician’s Global Assessment (PGS) questionnaire on days 28, 56, and 84 after injection, as described above. Patient satisfaction was assessed using the FACE-Q questionnaire prior to injection and on days 28, 56, and 84 after injection, as described above.

### Conclusions

Compound 1A low and high doses (averaged doses, of 80 mg and 158.5 mg, respectively) were safe and well tolerated with no clinically significant changes in vital signs. The most commonly reported AEs were bruising, induration edema, pain and erythema, all of which were reported with a similar incidence in the Compound 1A and placebo treatment groups. Edema and induration were reported with a higher severity in the Compound 1A high dose group compared to placebo, particularly in the first 24 hours following injection. All AEs were transient and did not require any treatment.

Based on the objective MRI data, the physician impression scale and subject FACEQ scale, it was concluded that Compound 1A was effective in submental fat reduction. This conclusion is supported by both the change in the submental thickness, as well as submental fat volume. The robustness of these results is supported by the dose response that shows larger effects for the higher dose of Compound 1A.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of reducing the quantity of adipose tissue in a subject in need thereof, comprising administering a plurality of injections of a pharmaceutical composition into an adipose portion of the arms, thighs, buttocks, abdomen, or submental region of the subject, wherein the pharmaceutical composition comprises:
a therapeutically effective amount of a compound of Formula (I): 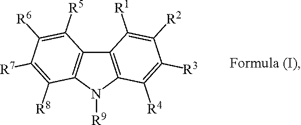
or a pharmaceutically-acceptable salt thereof, wherein:
each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary amino group or phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients;
optionally wherein said pharmaceutical composition comprises about 10% to about 30% water by weight.

2. The method of claim 1, wherein the plurality of injections is administered to an adipose portion of the submental region of the subject.

3. The method of claim 1 or 2, wherein the plurality of injections is provided in a specific grid pattern.

4. The method of any one of claims 1 to 3, wherein each of the plurality of injections into the adipose portion of the subject is provided at a different location of the adipose portion of the subject.

5. The method of any one of claims 1 to 4, wherein each of the plurality of injections into the adipose portion of the subject is provided to the arms, thighs, buttocks, or submental region of the subject.

6. The method of any one of claims 1 to 5, wherein the plurality of injections comprises between 20 and 60 injections.

7. The method of any one of claims 1 to 6, wherein the plurality of injections comprises between 40 and 50 injections.

8. The method of any one of claims 1 to 7, wherein the size of the grid is more than 20 points.

9. The method of any one of claims 1 to 8, wherein the size of the grid is more than 40 points.

10. The method of any one of claims 1 to 9, wherein the volume of each injection is between about 25 to about 250 microliters.

11. The method of any one of claims 1 to 10, wherein the volume of each injection is between about 25 to about 150 microliters.

12. The method of any one of claims 1 to 11, wherein the volume of each injection is between about 50 to about 100 microliters.

13. The method of any one of claims 1 to 12, wherein the concentration of the compound of Formula (I) in the pharmaceutical composition is between about 20 to about 100 milligrams per milliliter.

14. The method of any one of claims 1 to 13, wherein the concentration of the compound of Formula (I) in the pharmaceutical composition is between about 40 to about 70 milligrams per milliliter.

15. The method of any one of claims 1 to 14, wherein the concentration of the compound of Formula (I) in the pharmaceutical composition is about 50 milligrams per milliliter.

16. The method of any one of claims 1 to 15, wherein the total dose of the compound of Formula (I) per patient per sitting is less than 500 milligrams.

17. The method of any one of claims 1 to 16, wherein the total dose of the compound of Formula (I) per patient per sitting is less than 400 milligrams.

18. The method of any one of claims 1 to 17, wherein the total dose of the compound of Formula (I) per patient per sitting is less than 300 milligrams.

19. The method of any one of claims 1 to 18, wherein the total dose of the compound of Formula (I) per patient per sitting between about 0.5 milligrams to about 480 milligrams.

20. The method of any one of claims 1 to 19, wherein the total dose of the compound of Formula (I) per patient per sitting is between about 1 milligram to about 360 milligrams.

21. The method of any one of claims 1 to 20, wherein the total dose of the compound of Formula (I) per patient per sitting is between about 5 milligrams to about 240 milligrams.

22. The method of any one of claims 1 to 21, wherein the total dose of the compound of Formula (I) per patient per sitting is between about 10 milligrams to about 180 milligrams.

23. The method of any one of claims 1 to 22, wherein the total dose of the compound of Formula (I) per patient per sitting is between about 20 milligrams to about 120 milligrams.

24. The method of any one of claims 1 to 23, wherein the total dose of the compound of Formula (I) per patient per sitting is between about 40 milligrams to about 80 milligrams.

25. The method of any one of claims 1 to 24, wherein each injection comprises between about 1 milligram to about 25 milligrams of the compound of Formula (I).

26. The method of any one of claims 1 to 25, wherein each injection comprises between about 2.5 milligrams to about 15 milligrams of the compound of Formula (I).

27. The method of any one of claims 1 to 26, wherein each injection comprises between about 5 milligrams to about 10 milligrams of the compound of Formula (I).

28. The method of any one of claims 1 to 27, wherein R⁹ is C₁₋₉ alkyl substituted with at least one quaternary amino group.

29. The method of any one of claims 1 to 28, wherein the at least one quaternary amino group is of Formula (V): 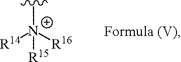
wherein each of R
¹⁴, R¹⁵, and R¹⁶ is independently C₁₋₉alkyl, C₂₋₉alkenyl, or C₂₋₉alkynyl.

30. The method of claim 29, wherein said at least one quaternary amino group has a general formula (V′): 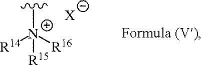
wherein X is a negatively charged ion.

31. The method of claim 30, wherein X is Cl.

32. The method of any one of claims 29-31, wherein each of R¹⁴, R¹⁵, and R¹⁶ is independently C₁₋₉alkyl.

33. The method of any one of claims 29-32, wherein each of R¹⁴, R¹⁵, and R¹⁶ is independently methyl.

34. The method of any one of claims 1 to 33, wherein at least one of R¹, R², R³, and R⁴ is halogen.

35. The method of any one of claims 1 to 33, wherein at least one of R⁵, R⁶, R⁷, and R⁸ is halogen.

36. The method of any one of claims 1 to 33, wherein at least one of R¹, R², R³, and R⁴ is halogen and at least one of R⁵, R⁶, R⁷, and R⁸ is halogen.

37. The method of any one of claims 34 to 36, wherein the halogen is bromo.

38. The method of any one of claims 1 to 33, wherein at least one of R¹, R², R³, and R⁴ is OH.

39. The method of any one of claims 1 to 33, wherein at least one of R⁵, R⁶, R⁷, and R⁸ is OH.

40. The method of any one of claims 1 to 33, wherein at least one of R¹, R², R³, and R⁴ is nitro and at least one of R⁵, R⁶, R⁷, and R⁸ is nitro.

41. The method of any one of claims 1 to 40, wherein the compound of Formula (I) is:
3-(3,6-dibromo-9H-carbazol-9-yl)-N,N,N-trimethylpropan-1-aminium,
5-(9H-carbazol-9-yl)-N,N,N-trimethylpentan-1-aminium,
5-(2-hydroxy-9H-carbazol-9-yl)-N,N,N-trimethylpentan-1-aminium, or
5-(3,6-dibromo-9H-carbazol-9-yl)-N,N,N-trimethylpentan-1-aminium.

42. The method of any one of claims 1 to 41, wherein said at least one compound of formula (I) is represented by the structure of formula (1): 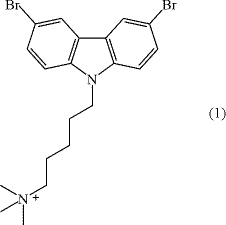 [SMILES: C[N+](C)(C)CCCCCn1c2ccc(Br)cc2c2cc(Br)ccc21]
.

43. The method of any one of claims 1-42, wherein the compound of Formula (I) is 5-(3,6-dibromo-9H-carbazol-9-yl)-N,N,N-trimethylpentan-1-aminium chloride.

44. The method of any one of claims 1 to 43, wherein said pharmaceutical composition comprises about 10% to about 30% water by weight.

45. The method of any one of claims 1 to 44, wherein the pharmaceutical composition comprises about 15% to about 30% water by weight.

46. The method of any one of claims 1 to 45, wherein the pharmaceutical composition comprises about 15% to about 25% water by weight.

47. The method of any one of claims 1 to 46, wherein the pharmaceutical composition comprises about 20% to about 30% water by weight.

48. The method of any one of claims 1 to 47, wherein the pharmaceutical composition comprises about 23% to about 27% water by weight.

49. The method of any one of claims 1 to 48, wherein the pharmaceutical composition comprises about 24% to about 26% water by weight.

50. The method of any one of claims 1 to 49, wherein the pharmaceutical composition comprises about 25% water by weight.

51. The method of any one of claims 1 to 50, wherein the pharmaceutical composition comprises more than one pharmaceutically acceptable excipient.

52. The method of any one of claims 1 to 51, wherein the one or more pharmaceutically acceptable excipients is selected from the group consisting of alcohols, fatty acids, ionic surfactants, nonionic surfactants, monoglycerides, diglycerides, triglycerides, esters, antioxidants, amino acids, and amino esters.

53. The method of claim 52, wherein the one or more pharmaceutically acceptable excipients is selected from the group consisting of propylene glycol, polyoxyl hydrogenated castor oil, ethanol, glycerol, sorbitol, mannitol, benzyl alcohol, lauryl glucoside, ammonium lauryl sulfate, sodium lauryl sulfate, sodium laureth sulfate, sodium myreth sulfate, docusate, perfluorooctanesulfonate, perfluorobutanesulfonate, sodium stearate, sodium lauroyl sarcosinate, perfluorononanoate, perfluorooctanoate, cetrimonium bromide, cetylpyridinium chloride, benzalkonium chloride, benzethonium chloride, dimethyldioctadecylammonium chloride, dimethyldioctadecylammonium bromide, 3-[(3-cholamidopropyl)dimethylammonio]-1-propanesulfonate, cocamidopropyl hydroxysultaine, cocamidopropyl betaine, phosphatidylserine, phosphatidylethanolamine, phosphatidylcholine, sphingomyelin, octaethylene glycol monododecyl ether, pentaethylene glycol monododecyl ether, nonaethylene glycol, polyethylene glycol nonyl phenyl ether, 2-[4-(2,4,4-trimethylpentan-2-yl)phenoxy]ethanol, Polyoxyl-35 castor oil, polyethoxylated tallow amine, cocamide monoethanolamine, cocamide diethanolamine, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, decyl glucoside, lauryl glucoside, octyl glucoside, lauryldimethylamine oxide, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneisosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, tetratriacontylic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, cervonic acid, herring acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, and citric acid.

54. The method of claim 53, wherein the one or more pharmaceutically acceptable excipients is selected from the group consisting of propylene glycol, polyoxyl hydrogenated castor oil, ethanol, glycerol, sorbitol, mannitol, benzyl alcohol, lauryl glucoside, ammonium lauryl sulfate, sodium lauryl sulfate, sodium laureth sulfate, sodium myreth sulfate, sodium stearate, sodium lauroyl sarcosinate, benzalkonium chloride, benzethonium chloride, phosphatidylcholine, sphingomyelin, 2-[4-(2,4,4-trimethylpentan-2-yl)phenoxy]ethanol, Polyoxyl-35 castor oil, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, lauric acid, tridecylic acid, myristic acid, palmitic acid, stearic acid, myristoleic acid, palmitoleic acid, sapienic acid, oleic acid, elaidic acid, vaccenic acid, gadoleic acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, and citric acid.

55. The method of claim 54, wherein the one or more pharmaceutically acceptable excipients is selected from the group consisting of ethanol, glycerol, propylene glycol, sorbitol, mannitol, benzyl alcohol, Polysorbate 20, Polysorbate 40, Polysorbate 60, and Polysorbate 80.

56. The method of any one of claims 1 to 55, wherein the pharmaceutical composition comprises at least about 0.1% by weight of the compound of Formula (I).

57. The method of any one of claims 1 to 56, wherein the pharmaceutical composition comprises between about 0.1% to about 10% by weight of the compound of Formula (I).

58. The method of any one of claims 1 to 57, wherein the pharmaceutical composition comprises between about 1% to about 5% by weight of the compound of Formula (I).

59. The method of any one of claims 1 to 58, further comprising an additional active agent.

60. The method of claim 59, wherein the additional active agent is an anti-inflammatory agent, an anti-fibrotic agent, prostaglandin E2, retinoic acid, or halofuginone.

61. The method of any one of claims 1 to 60, wherein the pharmaceutical composition is formulated for parenteral injection.

62. The method of any one of claims 1 to 61, wherein the pharmaceutical composition is formulated for subcutaneous injection.

63. The method of any one of claims 1 to 60, wherein the pharmaceutical composition is formulated for topical administration.

64. The method of any one of claims 1-63, wherein reducing the quantity of adipose tissue comprises reducing the volume of said adipose tissue.

65. The method of any one of claims 1-63, wherein reducing the quantity of adipose tissue comprises reducing the thickness of said adipose tissue.

66. The method of any one of claims 1-65, wherein reducing the quantity of adipose tissue comprises reducing the volume of submental fat.

67. The method of any one of claims 1-66, wherein reducing the quantity of adipose tissue comprises reducing the thickness of submental fat.

68. A pharmaceutical composition comprising:
a therapeutically effective amount of a compound of Formula (I): 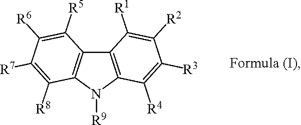
or a pharmaceutically-acceptable salt thereof, wherein:
each of R¹, R², R³, R⁴, R⁵, R⁶, R⁷, and R⁸ is independently H, halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), ON(═O), C₁₋₅alkyl, C₂₋₅alkenyl, or C₂₋₅alkynyl; wherein each alkyl, alkenyl, and alkynyl is independently optionally substituted with one or more substituents selected from the group consisting of halogen, —CN, —NO₂, -OR¹⁰, -SR¹⁰, -S(=O)R¹⁰, -S(=O)₂R¹⁰, -NR¹¹R¹², -C(=O)NR¹¹R¹², -S(=O)NR¹¹R¹², -S(=O)₂NR¹¹R¹², -C(=O)R¹⁰, -C(=O)OR¹⁰, -NR¹³C(=O)R¹⁰, -NR¹³C(=O)NR¹¹R¹², -NR¹³S(=O)₂R¹⁰, -NR¹³S(=O)₂NR¹¹R¹², -C(=S)R¹⁰, —N(═O), —SN(═O), -NR¹³N(=O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary amino group or phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or an R¹¹ and an R¹² may be taken together along with the nitrogen atom to which they are attached to form a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl; and
one or more pharmaceutically acceptable excipients;
optionally wherein said pharmaceutical composition comprises about 10% to about 30% water by weight,
for use in a method of reducing the quantity of adipose tissue in a subject in need thereof, wherein the pharmaceutical composition is in a unit dosage form suitable for administration by a plurality of injections into an adipose portion of the arms, thighs, buttocks, abdomen, or submental region of the subject.
